(19) United States
(12) Patent Application Publication
(10) Pub. No.: US 2024/0108505 A1
(43) Pub. Date: Apr. 4, 2024

(54) Surgical Irrigation Sleeve Formed of Polyether Block Amide Resin

(71) Applicant: Bausch & Lomb Ireland Limtied, Dublin (IE)

(72) Inventors: Nathan BELCHER, Kansas City, MO (US); Matthew J. FITZGERALD, St. Louis, MO (US)

(73) Assignee: Bausch & Lomb Ireland Limtied, Dublin (IE)

(21) Appl. No.: 18/376,698

(22) Filed: Oct. 4, 2023

Related U.S. Application Data
(60) Provisional application No. 63/413,042, filed on Oct. 4, 2022.

(51) Int. Cl. A61F 9/007; A61L 29/06

(52) U.S. Cl. CPC A61F 9/00745; A61L 29/06; A61B 2217/005; A61B 2217/007

(57) ABSTRACT

An ophthalmic surgical irrigation assembly for attachment to an ophthalmic surgical instrument. The assembly includes an elongated sleeve having a hollow interior space through which fluid is passed and surrounds a majority length of a cannula attached to the ophthalmic surgical instrument. A fluid port formed adjacent a distal end of the elongated sleeve allows the fluid to exit the interior space. A distal opening in the elongated sleeve allows a distal portion of the cannula to extend beyond the distal opening. A hub integrally formed with a proximal end of the elongated sleeve is adapted for fluid tight attachment to at least a portion of the surgical instrument and allows fluid to flow from the surgical instrument through the interior space and exit out of the fluid port. The elongated sleeve and the hub are each formed of a polyether block amide resin.

### FIELD

The present disclosure relates to surgical irrigation sleeves, particularly to coaxial ophthalmic surgical irrigation sleeves for providing irrigating fluids into an eye during surgery.

### BACKGROUND

This section provides background information related to the present disclosure which is not necessarily prior art.

The use of coaxial irrigation sleeves in ophthalmic surgery is well known. For example, silicone sleeves have been in use for decades and provide good wound sealing properties as well as thermal insulating properties. The thermal insulating properties protect the eye tissue from thermal damage, sometimes referred to as corneal burns, while a ultrasonic Phaco needle is vibrated during surgery.

While the known silicone sleeves can be effective, one major drawback is that the supple sleeves tend to collapse when a surgeon attempts to insert the sleeve and needle through the eye incision. To combat sleeve collapse the prior art has added ribbed structure along the sleeve or provided a rigid inner layer to stiffen the sleeve. Other sleeve collapse avoidance techniques have included a tight-fit between a thin sleeve distal end and the needle.

It is also known to form an irrigation sleeve out of hard, thermoplastic polyurethanes, polyesters, or polyolefins as disclosed in U.S. Pat. No. 4,897,079. However, the sleeve materials disclosed have a durometer of at least 60 Shore D, which is quite hard and is not likely to create an effective seal with the eye incision.

It would be desirable to provide an irrigation sleeve that is soft enough to sufficiently collapse to create a seal with eye incision but yet hard enough to not collapse upon insertion through the eye incision.

### DRAWINGS

The drawings described herein are for illustrative purposes only of selected embodiments and not all possible implementations, and are not intended to limit the scope of the present disclosure.

FIG. 1 is an elevation view of prior art irrigation sleeve showing the sleeve collapsing at the eye incision; and

FIG. 2 is an elevation view of an example irrigation sleeve in accordance with the present invention.

Corresponding reference numerals indicate corresponding parts throughout the several views of the drawings.

### DETAILED DESCRIPTION

Example embodiments will now be described more fully with reference to the accompanying drawings.

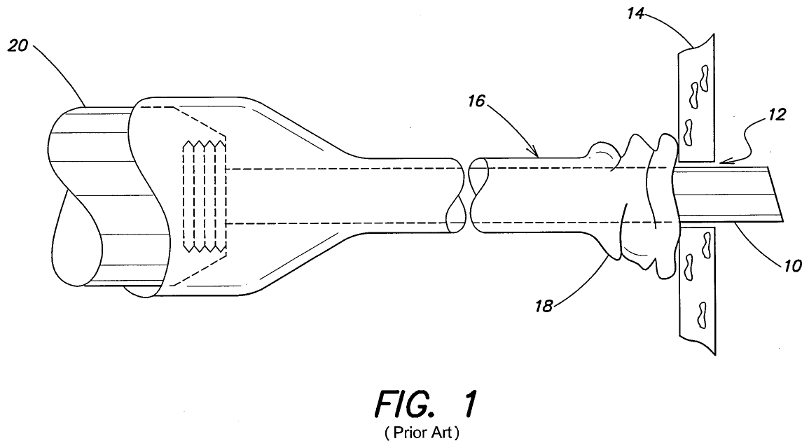

Current trends in ophthalmic surgery are demanding ever smaller diameter needles and smaller incisions. It is believed that smaller incisions lead to less change in corneal curvature and better visual acuity post-operation. These smaller incisions have led to tighter interfaces with the sleeves and needles. A problem, becoming more prevalent, is that the prior art silicone sleeve tends to accordion, i.e. collapse, at the incision, as the surgeon attempts to insert the sleeve and needle through the incision. This prior art problem is shown at FIG. 1. A needle 10 has passed through an incision 12 in an eye 14, but a sleeve 16 has accordioned at 18 outside the eye 14. Needle 10 may be attached to a phaco handpiece 20 in a known manner, typically via a threaded connection, as shown. It is critical that sleeve 16 pass through incision 12 and into the eye 14 along with needle 10, so that sleeve 16 can protect the eye tissue from damage and to provide irrigation fluid into the eye 14.

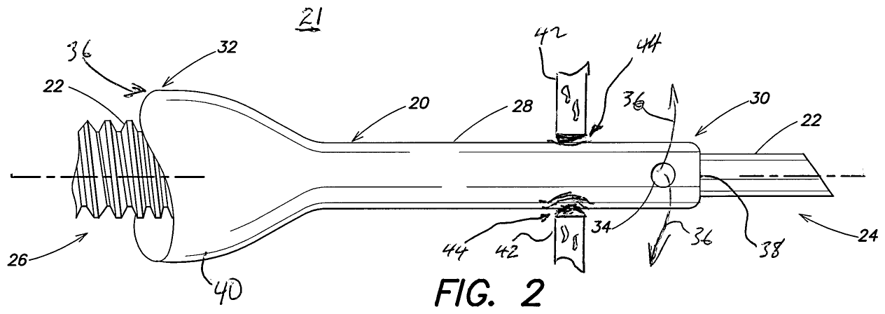

FIG. 2 shows an ophthalmic surgical irrigation assembly 21, in accordance with the present disclosure. Assembly 21 may include an elongated sleeve 20 having a hollow interior space (not shown) through which fluid 36 passes. Sleeve 20 coaxially surrounds a majority length of a cannula 22 having a distal end portion 24 and a proximal end 26. Proximal end 26 is typically attached to a conventional phacoemulsification handpiece (not shown but essentially the same as in FIG. 1) and the handpiece may include a port (not shown) for delivering irrigation fluid 36 between the sleeve 20 and cannula 22. Sleeve 20 includes an elongated, resilient essentially tubular portion 28 having a distal end 30 and a proximal end 32 for surrounding a majority portion of a shaft of the cannula 22. At lease one fluid port 34 (though typically at least two fluid ports) is formed adjacent the sleeve distal end 30 for allowing the fluid 36 to exit the interior space. A distal opening (indicated by 38) is formed in the sleeve 20 and allows the distal end portion 24 of the cannula 22 to extend beyond the distal opening 38. Sleeve proximal end 32 includes an enlarged hub 40, as shown, integrally formed with the sleeve 20 at a proximal end of tubular portion 28. The hub 40 is adapter for fluid tight attachment to at least a portion of the surgical instrument for allowing the fluid 36 to flow from the surgical instrument through the interior space and exit out of the fluid port 34. The elongated sleeve 20 and the hub40 are each formed of a polyether block amide resin to reduce the risk of the elongated sleeve collapsing at the eye incision.

The polyether block amide resin allows elongated sleeve 20 along tubular portion 28 to be compressible by an eye incision 42, after insertion through the eye incision 42 for forming a fluid seal (shown at 44) to reduce fluid leakage at the eye incision. The polyether block amide resin enables the elongated sleeve 20 to not be substantially collapsed by the eye incision 44 during normal surgical use, thus allowing a continuous flow of fluid 36 out of the fluid port 34.

The sleeve distal end 30 may have a close-fit proximate cannula distal end 24 for retaining the sleeve distal end 30 near the needle distal end 24.

Preferably, the irrigation assembly is molded as a single unit. Also, the ophthalmic surgical instrument may be a phacoemulsification instrument, as described above, or may be an irrigation/aspiration instrument (not shown).

Preferably, the polyether block amide resin has a durometer of about 95 Shore A or less and more preferably 90 Shore A or less. It has been discovered that such a durometer is soft enough to form an effective seal at the eye incision yet hard enough not to collapse the sleeve outside the eye incision while attempting insertion through the eye incision and for the sleeve not to be collapsed by the eye incision after insertion. The polyether block amide resin also preferably has a flexural modulus of at least about 10,000 pounds per square inch (psi) (about 703 kg/cm 2) and more preferably at least 12,200 psi (about 858 kg/cm 2). The polyether block amide (PEBA) resin may be marketed by Arkema Inc. as PEBAX® 4033 SA 01 Med or by Evonik Industries under the brand VESTAMID®E brand.

The PEBA resin described in this specification is believed to provide superior combined wound sealing and stiffness for insertion through the eye incision compared to the prior art polyurethanes, polyesters, or polyolefins as disclosed in U.S. Pat. No. 4,897,079. The materials disclosed in the '079 patent are significantly harder (60-90 Shore D) than those of the present invention (90-95 Shore A, roughly equivalent to 35-46 Shore D). In this way, the present invention provides a better seal at the eye incision while being stiff enough for insertion but yet more flexible than (not a hard as) the '079 sleeve.

A sleeve in accordance with the present invention by also be formed of other Nylon materials that result in the unique improvements for sleeves described above, including resistance to tearing, twisting, and buckling while retaining it's ability to conform to the incision to seal and prevent wound leakage. PEBA sleeves, as described above, also offer improved function for angled phaco needles addressing some of the bunching and buckling issues common to the angled needle application. These improvements are aided by use of a higher durometer than typically available in common prior art silicone sleeves but of a lower durometer than other prior art sleeve materials.

In addition to the preferred durometer range, the stiffness properties combine with the low surface friction in a manner that was not foreseen and offers sleeve performance that is a major advancement beyond the current silicone sleeves.

The PEBA resin disclosed above and other nylon materials have the advantage of being able to form sleeves that are easier to net mold and provide better overall performance in the eye compared with prior art surgical irrigation sleeves.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. An ophthalmic surgical irrigation assembly for attachment to an ophthalmic surgical instrument comprising:
an elongated sleeve having a hollow interior space through which fluid is passed and for surrounding a majority length of a cannula attached to the ophthalmic surgical instrument;
at least one fluid port formed adjacent a distal end of the elongated sleeve for allowing the fluid to exit the interior space;
a distal opening formed in the elongated sleeve for allowing a distal end portion of the cannula to extend beyond the distal opening;
a hub integrally formed with a proximal end of the elongated sleeve and adapted for fluid tight attachment to at least a portion of the surgical instrument for allowing fluid to flow from the surgical instrument through the interior space and exit out of the fluid port; and
wherein the elongated sleeve and the hub are each formed of a polyether block amide resin.

2. The sleeve of claim 1, wherein the polyether block amide resin has a durometer of about 95 Shore A or less.

3. The sleeve of claim 1, wherein the polyether block amide resin has a durometer of 90 Shore A or less.

4. The sleeve of claim 1, wherein the elongated sleeve is compressible by an eye incision after insertion through the eye incision for forming a fluid seal to reduce fluid leakage at the eye incision.

5. The sleeve of claim 4, wherein the elongated sleeve is not substantially collapsed by the eye incision during normal surgical use, thus allowing a continuous flow of fluid out of the fluid port.

6. The sleeve of claim 1, wherein the irrigation assembly is molded as single unit.

7. The sleeve of claim 1, wherein the polyether block amide resin is PEBAX® 4033 SA 01 Med.

8. The sleeve of claim 1, has a flexural modulus of at least about 10,000 pounds per square inch.

8. The sleeve of claim 1, wherein the ophthalmic surgical instrument is a phacoemulsification instrument.

9. The sleeve of claim 1, wherein the ophthalmic surgical instrument is an irrigation/aspiration instrument.
